(19) United States
(12) Patent Application Publication
(10) Pub. No.: US 2013/0109746 A1
(43) Pub. Date: May 2, 2013

(54) COMPOSITION COMPRISING ELLAGIC ACID AND A PARTICULAR CATIONIC SURFACTANT, AND COSMETIC USE THEREOF

(71) Applicants: Tiphaine Derkx, Paris (FR); Laurence Richet, Presles (FR)

(72) Inventors: Tiphaine Derkx, Paris (FR); Laurence Richet, Presles (FR)

(73) Assignee: L'OREAL S.A., Paris (FR)

(21) Appl. No.: 13/581,632

(22) PCT Filed: Feb. 28, 2011
(86) PCT No.: PCT/EP2011/052933

§ 371 Date: Aug. 29, 2012

Related U.S. Application Data
(60) Provisional application No. 61/309,535, filed on Mar. 2, 2010.

(30) Foreign Application Priority Data
Mar. 1, 2010 (FR) 1051444

(51) Int. Cl. A61K 8/49; A61Q 5/00

(52) U.S. Cl. CPC A61K 8/498; A61Q 5/006
USPC 514/453

(57) ABSTRACT

The invention relates to a cosmetic or dermatological composition comprising, in a cosmetically acceptable medium:

(i) one or more compounds chosen from ellagic acid, ethers thereof and ellagic acid salts or ether salts thereof, and

(ii) one or more cationic surfactants comprising one or more permanent positive charges.

The invention also relates to a cosmetic process for treating keratin materials with this composition, especially for the treatment of dandruff.

The present invention relates to a cosmetic or dermatological composition comprising ellagic acid or a derivative thereof and at least one particular surfactant.

Ellagic acid is a compound in the form of a white powder, with a melting point above 360° C. It is an active agent that is known in the field of cosmetics for its use as an anti-canities active agent (EP 1 870 081), antioxidant, depigmenting agent (U.S. Pat. No. 5,073,545) or antipollution agent (EP 1 282 395).

It may also be used as an antidandruff agent.

Dandruff problems affect up to 50% of the world's population. They affect both men and women and are perceived as having a very negative psychosocial impact. The appearance of dandruff is disagreeable both aesthetically and because of the discomfort it causes (itching, redness . . . ), and as such many people confronted with this problem to variable degrees wish to eliminate it efficiently and definitively.

Dandruff corresponds to excessive and visible desquamation of the scalp resulting from excessively rapid multiplication of the epidermal cells and their abnormal maturation. This phenomenon may be caused especially by microtrauma of physical or chemical nature, such as excessively aggressive hair treatments, extreme climatic conditions, nervousness, the diet, fatigue and pollution, but it has been demonstrated that dandruff conditions usually result from a disorder of the microflora of the scalp and are more particularly due to the excessive colonization of a fungus belonging to the family of yeasts of the genus Malassezia (previously known as Pityrosporum) and which is naturally present on the scalp.

There is thus great interest in upgrading this ingredient in suitable formulations. However, since ellagic acid is very sparingly soluble in water and in the solvents and oils commonly used in cosmetic compositions, it is thus difficult to incorporate it uniformly into cosmetic compositions, especially into compositions that comprise an aqueous medium, without giving rise to the formation of aggregates that might cause instability of the composition over time and a loss of efficacy.

Now, such an unstable composition cannot be applied to the skin and/or the hair since it cannot distribute the ellagic acid efficiently over the treated zones. The efficacy of ellagic acid is thus compromised.

Moreover, it is known practice from patent application FR-A-2 855 051 to disperse ellagic acid powder using a salt of a copolymer of maleic anhydride and of diisobutylene and a copolymer of polyvinylpyrrolidone type, using a milling technique with the aid of a ball mill, which requires long and expensive operations to make this dispersion.

Patent application FR-A-2 893 505 describes an aqueous dispersion of ellagic acid made with a copolymer consisting of polyethylene glycol and polypropylene glycol blocks. However, it has been found that these aqueous dispersions do not show good stability over time, especially after 24 hours of storage at room temperature (25° C.), since the ellagic acid particles fall to the bottom of the water and no longer remain in suspension. The aqueous dispersion is thus no longer homogeneous after a certain storage time, making it unsuitable for use for the manufacture of cosmetic products.

There is thus a need for aqueous compositions of ellagic acid or of a derivative thereof that promote the dispersion of ellagic acid or of the derivative and that limit the formation of aggregates and allow very good distribution of the active agent during application and thus optimum efficacy, while at the same time conserving good working properties and good cosmetic properties.

More specifically, a subject of the invention is a cosmetic or dermatological composition comprising, in a cosmetically acceptable medium:

(i) one or more compounds chosen from ellagic acid, ethers thereof and ellagic acid salts or ethers thereof, and

(ii) one or more cationic surfactants comprising one or more permanent positive charges.

Such a composition spreads easily on the hair and the scalp, is removed easily with water, and gives clean, shiny, soft hair.

The composition according to the invention shows good homogeneity and good stability on storage both at room temperature (25° C.) and at higher temperatures (37 or 45° C., for example) and does not lead to the formation of aggregates.

A subject of the invention is also a non-therapeutic cosmetic treatment process for treating keratin materials, especially the hair and the scalp, comprising the application to the said materials of a composition as described previously. The cosmetic treatment process is in particular a process for eliminating and/or reducing dandruff, and especially that caused by yeasts of the genus Malassezia, comprising the application of a cosmetically effective amount of the said dispersion as an antidandruff cosmetic agent to the scalp and/or the hair.

The composition according to the invention comprises one or more compounds (i) chosen from ellagic acid, ethers thereof and ellagic acid salts or ethers thereof.

Ellagic acid, also known as 2,3,7,8-tetrahydroxy(1)benzopyrano(5,4,3-cde)(1)benzopyran-5,10-dione, is a well-known molecule belonging to the polyphenol group, and is present in the plant kingdom. Reference may be made to the Merck Index 20th edition (1996), No. 3588.

Ellagic acid has the chemical formula (I) below:

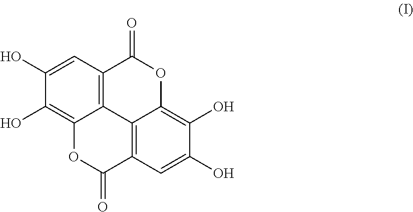 [SMILES: O=c1oc2c(O)c(O)cc3c(=O)oc4c(O)c(O)cc1c4c23]

which comprises four fused rings.

Ellagic acid is commercially available, for example from the company Sigma, France.

Document FR-A-1 478 523 discloses a process for purifying ellagic acid and also the purified ellagic acids obtained via such a process.

The ellagic acid ether(s) that may be used according to the invention are preferably chosen from the mono-, di-, tri- or polyethers obtained by etherification of one or more hydroxyl groups (one of the four OH groups of ellagic acid) with one or more groups chosen from C₂-C₂₀ alkyl groups, polyoxyalkylene groups or groups derived from one or more mono- or polysaccharides.

Such ethers are described in patent U.S. Pat. No. 5,073,545. Preferably, these ellagic acid ethers are chosen from 3,4-di-O-methylellagic acid, 3,3′,4-tri-O-methylellagic acid and 3,3′-di-O-methylellagic acid.

The ellagic acid salt(s) that may be used according to the invention are preferably chosen from the salts of alkali metals or alkaline-earth metals, such as sodium, potassium, calcium and magnesium, ammonium salts and amine salts such as triethanolamine, monoethanolamine, arginine and lysine salts. Preferably, the ellagic acid salt(s) or ethers thereof that may be used according to the invention are chosen from the salts of alkali metals or alkaline-earth metals, especially the sodium, potassium, calcium or magnesium salts.

Among all the compounds (i) mentioned, it is preferred to use ellagic acid and salts thereof.

Ellagic acid and its ethers or ellagic acid salts or ether salts thereof may be present in the cosmetic or dermatological composition according to the invention in a content ranging from 0.01% to 10% by weight, in particular from 0.05% to 5%, preferably from 0.1% to 5%, and better still from 0.2 to 2% by weight relative to the total weight of the cosmetic composition.

The composition according to the invention comprises one or more cationic surfactants.

In general, a cationic surfactant may bear one or more positive permanent charges or may contain one or more cationizable functions in the composition according to the invention. The cationic surfactants of the invention are those bearing one or more positive permanent charges, i.e. one or more quaternized nitrogen atoms.

As examples of cationic surfactants that may be used in the cosmetic composition according to the invention, mention may be made especially of quaternary ammonium salts, such as quaternary ammonium salts that have the general formula (II) below:

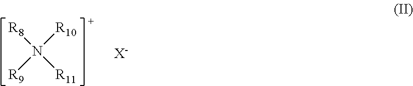

in which the radicals R₈ to R₁₁, which may be identical or different, represent a linear or branched aliphatic radical comprising from 1 to 30 carbon atoms or an aromatic radical such as aryl or alkylaryl, at least one of the radicals R₈ to R₁₁ comprising from 8 to 30 carbon atoms and preferably from 12 to 24 carbon atoms. The aliphatic radicals may comprise heteroatoms especially such as oxygen, nitrogen, sulfur and halogens.

The aliphatic radicals are chosen, for example, from C₁₋₃₀ alkyl, C₁₋₃₀ alkoxy, polyoxyalkylene (C₂-C₆), C₁₋₃₀ alkylamide, (C₁₂-C₂₂)alkylamido(C₂-C₆)alkyl, (C₁₂-C₂₂)alkylacetate, and C₁₋₃₀ hydroxyalkyl; X⁻ is an anion chosen from the group of halides, phosphates, acetates, lactates, (C₁-C₄)alkyl sulfates and (C₁-C₄)alkyl- or (C₁-C₄)alkylaryl-sulfonates.
quaternary ammonium salts of imidazoline, for instance those of formula (III) below:

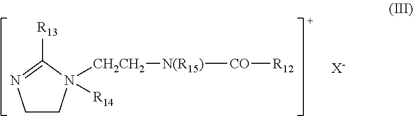

in which R₁₂ represents an alkenyl or alkyl group comprising from 8 to 30 carbon atoms, for example fatty acid derivatives of tallow; R₁₃ represents a hydrogen atom, a C₁-C₄ alkyl group or an alkenyl or alkyl group comprising from 8 to 30 carbon atoms; R₁₄ represents a C₁-C₄ alkyl group; R₁₅ represents a hydrogen atom, a C₁-C₄ alkyl group; X⁻ is an anion chosen from the group of halides, phosphates, acetates, lactates, (C₁-C₄)alkyl sulfates and (C₁-C₄)alkyl- or (C₁-C₄)alkylarylsulfonates.

R₁₂ and R₁₃ preferably denote a mixture of alkenyl or alkyl groups comprising from 12 to 21 carbon atoms, for example tallow fatty acid derivatives, R₁₄ denotes a methyl group, R₁₅ denotes a hydrogen atom. Such a product is, for example, sold under the name Rewoquat® W 75 by the company Rewo;

Among the quaternary ammonium salts of formula (II), the ones preferably used are, on the one hand, tetraalkylammonium chlorides, for instance dialkyldimethylammonium chlorides or alkyltrimethylammonium chlorides, in which the alkyl radical comprises from about 12 to 22 carbon atoms, in particular behenyltrimethylammonium chloride, distearyldimethylammonium chloride, cetyltrimethylammonium chloride, or benzyldimethylstearylammonium chloride, or, on the other hand, distearoylethylhydroxyethylmethylammonium methosulfate, dipalmitoylethylhydroxyethylammonium methosulfate or distearoylethylhydroxyethylammonium methosulfate, or else, finally, palmitylamidopropyltrimethylammonium chloride or stearamidopropyldimethyl(myristyl acetate)ammonium chloride sold under the name Ceraphyl® 70 by the company Van Dyk.
quaternary di- or triammonium salts in particular of formula (IV):

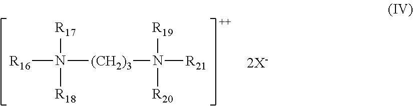

in which R16 denotes an alkyl radical comprising approximately 16 to 30 carbon atoms, optionally hydroxylated and/or interrupted with one or more oxygen atoms, R17 is chosen from hydrogen, an alkyl radical comprising from 1 to 4 carbon atoms and a group [(R16a)(R17a)(R18a)N⁺-(CH2)3-, Y⁻], R16a, R17a, R18a, R18, R19, R20 and R21, which may be identical or different, are chosen from hydrogen and an alkyl radical comprising from 1 to 4 carbon atoms, and X⁻ and Y—, which may be identical or different, are halides, acetates, phosphates, nitrates, (C₁-C₄)alkyl sulfates, (C₁-C₄)alkyl-sulfonate or (C₁-C₄)alkylaryl-sulfonates, in particular methyl sulfate or ethyl sulfate. Such compounds are, for example, Finquat CT-P sold by the company Finetex (Quaternium-89) and Finquat CT sold by the company Finetex (Quaternium-75),
quaternary ammonium salts containing at least one ester function, such as those of formula (V) below:

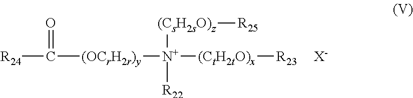

in which:

R₂₂ is chosen from C₁-C₆ alkyl and C₁-C₆ hydroxyalkyl or dihydroxyalkyl radicals;
R₂₃ is chosen from:

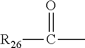

the radical
radicals R₂₇, which are linear or branched, saturated or unsaturated C₁-C₂₂ hydrocarbon-based radicals,
a hydrogen atom,
R₂₅ is chosen from:
the radical

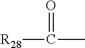

radicals R₂₉, which are linear or branched, saturated or unsaturated C₁-C₆ hydrocarbon-based radicals,
a hydrogen atom,

R₂₄, R₂₆ and R₂₈, which may be identical or different, are chosen from linear or branched, saturated or unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or mineral anion;

with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, but more particularly linear.

R₂₂ preferably denotes a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide), a (C₁-C₄)alkyl sulfate or a (C₁-C₄)alkyl- or (C₁-C₄)alkylaryl-sulfonate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride, methyl sulfate and or ethyl sulfate.

Use is made more particularly in the composition according to the invention of the ammonium salts of formula (V) in which:
R₂₂ denotes a methyl or ethyl radical,
x and y are equal to 1;
z is equal to 0 or 1;
r, s and t are equal to 2;
R₂₃ is chosen from:
the radical

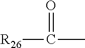

methyl, ethyl or C₁₄-C₂₂ hydrocarbon-based radicals,
a hydrogen atom;
R₂₅ is chosen from:
the radical

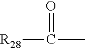

a hydrogen atom;
R₂₄, R₂₆ and R₂₈, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (V) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl paratoluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention preferably contains a mixture of quaternary ammonium salts of mono-, di- and triesters with a weight majority of diester salts.

Examples of mixtures of ammonium salts that may be used include the mixture containing 15% to 30% by weight of acyloxyethyldihydroxyethylmethylammonium methyl sulfate, 45% to 60% of diacyloxyethylhydroxyethylmethylammonium methyl sulfate and 15% to 30% of triacyloxyethylmethylammonium methyl sulfate, the acyl radicals containing from 14 to 18 carbon atoms and being derived from palm oil that is optionally partially hydrogenated.

It is also possible to use the ammonium salts containing at least one ester function that are described in patents U.S. Pat. No. 4,874,554 and U.S. Pat. No. 4,137,180.

Among the quaternary ammonium salts of formula (I), the ones preferably used are, on the one hand, tetraalkylammonium chlorides, for instance dialkyldimethylammonium chlorides or alkyltrimethylammonium chlorides, in which the alkyl radical comprises from about 12 to 22 carbon atoms, in particular behenyltrimethylammonium chloride, distearyldimethylammonium chloride, cetyltrimethylammonium chloride, or benzyldimethylstearylammonium chloride, or, on the other hand, distearoylethylhydroxyethylmethylammonium methosulfate, dipalmitoylethylhydroxyethylammonium methosulfate or distearoylethylhydroxyethylammonium methosulfate, or else, finally, palmitylamidopropyltrimethylammonium chloride or stearamidopropyldimethyl(myristyl acetate)ammonium chloride sold under the name Ceraphyl® 70 by the company Van Dyk.

Among all the cationic surfactants that may be present in the composition according to the invention, the ones preferably chosen are cetyltrimethylammonium salts, behenyltrimethylammonium salts, dipalmitoylethylhydroxyethylmethylammonium salts, distearoylethylhydroxyethylmethylammonium salts and methyl(C₉-C₁₉)alkyl(C₁₀-C₂₀)alkylamidoethylimidazolium salts, and mixtures thereof.

The cationic surfactant(s) may be present in a content ranging from 0.01% to 15% by weight relative to the total weight of the cosmetic or dermatological composition, preferably ranging from 0.1% to 10% by weight and more preferentially from 0.2% to 5% by weight.

Preferably, the cationic surfactant(s) (ii)/compound(s) (1) weight ratio ranges from 0.1 to 50, even more preferentially from 0.5 to 25, better still from 1 to 10 and even better still from 1 to 5.

The cosmetically acceptable medium is formed from water or from a mixture of water and of at least one cosmetically acceptable solvent preferably chosen from C₁-C₄ lower alcohols, such as ethanol, isopropanol, tert-butanol or n-butanol; polyols such as glycerol, propylene glycol and polyethylene glycols; and mixtures thereof.

In the composition according to the invention, the water may be present in a content ranging from 10% to 95% by weight and preferably ranging from 20% to 95% by weight relative to the total weight of the composition.

The composition according to the invention may also comprise one or more thickeners.

For the purposes of the present invention, the term “thickener” means an agent which, when introduced at 1% by weight in an aqueous solution or an aqueous-alcoholic solution containing 30% ethanol, and at pH 7, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like).

The thickener(s) may be chosen from fatty acid amides obtained from C10-C30 carboxylic acids (coconut acid monoisopropanol-, diethanol- or monoethanolamide, oxyethylenated carboxylic acid monoethanolamide alkyl ether), nonionic cellulose-based thickeners (hydroxyethylcellulose, hydroxypropylcellulose, carboxymethylcellulose), guar gum and nonionic derivatives thereof (hydroxypropyl guar), gums of microbial origin (xanthan gum, sclerogiucan gum), crosslinked homopolymers and copolymers based on acrylic acid or methacrylic acid or acrylamidopropanesulfonic acid and the associative polymers as described below.

The associative polymer(s) that may be used according to the invention are water-soluble polymers that are capable, in aqueous medium, of reversibly combining with each other or with other molecules.

Their chemical structure comprises hydrophilic zones and hydrophobic zones characterized by at least one fatty chain preferably comprising from 10 to 30 carbon atoms.

The associative polymer(s) that may be used according to the invention may be of anionic, cationic, amphoteric or nonionic type, such as the polymers sold under the names Pemulen TR1 or TR2 by the company Goodrich (INCI: Acrylates/C10-30 Alkyl Acrylate Crosspolymer), Salcare SC90 by the company Ciba, Aculyn 22, 28, 33, 44 or 46 by the company Röhm & Haas and Elfacos T210 and T212 by the company Akzo.

Among all the thickeners mentioned, use is preferably made of homopolymers and copolymers based on acrylic acid or methacrylic acid, which are preferably crosslinked (add the preference in B094751FR) the fatty acid amides obtained from a C10-C30 carboxylic acid.

Preferably, the cosmetic composition comprises from 0.1% to 20% by weight and better still from 0.2% to 10% by weight of thickener(s) relative to the total weight of the composition.

The composition according to the invention may also comprise one or more conditioning agents other than the cationic surfactants (ii).

According to the present invention, the term “conditioning agent” denotes any compound that can improve the cosmetic properties of the hair, in particular the softness, disentangling, feel and static electricity.

Preferably, the conditioning agent is chosen from the group of cationic polymers, organosiloxanes, linear or branched C8-C30 hydrocarbons, linear or branched C8-C30 fatty alcohols, esters of a C8-C30 fatty acid and of a C1-C30 alcohol, esters of a C1-C7 acid or diacid and of a C8-C30 fatty alcohol, esters of a C8-C30 fatty acid and of a C8-C30 fatty alcohol, ceramides or ceramide analogues, and mixtures of these compounds.

The term “cationic polymer” means a polymer that is positively charged when it is contained in the composition according to the invention. This polymer may bear one or more positive permanent charges or may contain one or more cationizable functions in the composition according to the invention.

The cationic polymer(s) that may be used as conditioning agents according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1000 and 3 000 000.

When the conditioning agent is a cationic polymer, it is preferably chosen from those that contain units comprising primary, secondary, tertiary and/or quaternary amine groups that may either form part of the main polymer chain, or may be borne by a side substituent directly attached thereto.

Among the cationic polymers that may be mentioned more particularly are polymers of the polyamine, polyamino amide and polyquaternary ammonium type. These are known products. They are described, for example, in French patents 2 505 348 and 2 542 997.

Among these polymers, mention may be made of:

(1) homopolymers or copolymers which are derived from acrylic or methacrylic esters or amides and comprise at least one of the units of the following formulae:

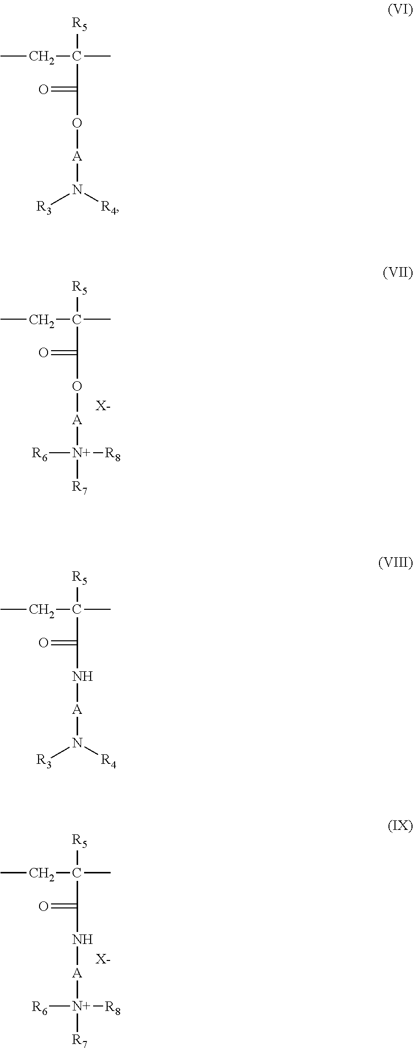

in which:

R3 and R4, which may be identical or different, represent hydrogen or an alkyl group containing from 1 to 6 carbon atoms and preferably methyl or ethyl;

R5, which may be identical or different, denote a hydrogen atom or a CH3 radical;

A, which may be identical or different, represent a linear or branched alkyl group of 1 to 6 carbon atoms, preferably 2 or 3 carbon atoms, or a hydroxyalkyl group of 1 to 4 carbon atoms;

R6, R7 and R8, which may be identical or different, represent an alkyl group containing from 1 to 18 carbon atoms or a benzyl radical and preferably an alkyl group containing from 1 to 6 carbon atoms;

X denotes an anion derived from a mineral or organic acid, such as a methosulfate anion or a halide such as chloride or bromide.

The copolymers of family (1) can also contain one or more units derived from comonomers which may be chosen from the family of acrylamides, methacrylamides, diacetone acrylamides, acrylamides and methacrylamides substituted on the nitrogen with lower (C1-C4) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxyethyltrimethylammonium chloride described, for example, in patent application EP-A-080 976 and sold under the name Bina Quat P 100 by the company Ciba Geigy,
the copolymer of acrylamide and of methacryloyloxyethyltrimethylammonium methosulfate sold under the name Reten by the company Hercules,

quaternized or non-quaternized vinylpyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,

dimethylaminoethyl methacrylate/vinylcaprolactam/vinylpyrrolidone terpolymers, such as the product sold under the name Gaffix VC 713 by the company ISP,

vinylpyrrolidone/methacrylamidopropyldimethylamine copolymers sold in particular under the name Styleze CC 10 by ISP,
quaternized vinylpyrrolidone/dimethylaminopropylmethacrylamide copolymers such as the product sold under the name Gafquat HS 100 by the company ISP, and
crosslinked polymers of methacryloyloxyalkyl(C1-C4)trialkyl(C1-C4)ammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.

(2) polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are especially described in French patents 2 162 025 and 2 280 361.

(3) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bisunsaturated derivative, a bis-halohydrin, a bisazetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are especially described in French patents 2 252 840 and 2 368 508.

(4) polyamino amide derivatives resulting from the condensation of polyalkylene polyamines with polycarboxylic acids followed by alkylation with difunctional agents. Mention may be made, for example, of adipic acid/dialkylaminohydroxyalkyl-dialkylene-triamine polymers in which the alkyl radical contains from 1 to 4 carbon atoms and preferably denotes methyl, ethyl or propyl. Such polymers are especially described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The mole ratio between the polyalkylene polyamine and the dicarboxylic acid is between 0.8:1 and 1.4:1; the polyamino amide resulting therefrom is reacted with epichlorohydrin in a mole ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(6) cyclopolymers of alkyldiallylamine or of dialkyldiallylammonium, such as the homopolymers or copolymers containing, as main constituent of the chain, units corresponding to formula (X) or (XI):

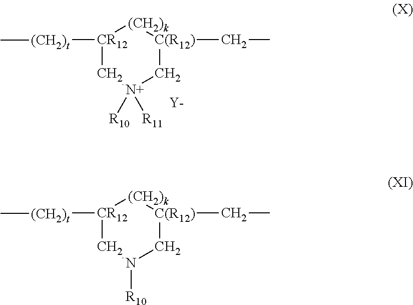

in which formulae k and t are equal to 0 or 1, the sum k+t being equal to 1; R12 denotes a hydrogen atom or a methyl radical; R10 and R11, independently of one another, denote an alkyl group having from 1 to 6 carbon atoms, a hydroxyalkyl group in which the alkyl group preferably has 1 to 5 carbon atoms, a lower (C1-C4) amidoalkyl group, or R10 and R11 may denote, together with the nitrogen atom to which they are attached, heterocyclic groups, such as piperidinyl or morpholinyl; Y⁻ is an anion such as bromide, chloride, acetate, borate, citrate, tartrate, bisulfate, bisulfite, sulfate or phosphate. These polymers are especially described in French patent 2 080 759 and in its Certificate of Addition 2 190 406.

R10 and R11, independently of one another, preferably denote an alkyl group having from 1 to 4 carbon atoms.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) the quaternary diammonium polymer containing repeating units corresponding to the formula:

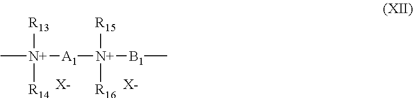

in which formula (XII):

R13, R14, R15 and R16, which may be identical or different, represent aliphatic, alicyclic or arylaliphatic groups containing from 1 to 20 carbon atoms, or lower hydroxyalkylaliphatic groups, or else R13, R14, R15 and R16, together or separately, constitute, with the nitrogen atoms to which they are attached, heterocycles optionally containing a second heteroatom other than the nitrogen, or else R13, R14, R15 and R16 represent a linear or branched C1-C6 alkyl group which is substituted with a nitrile, ester, acyl, amide or CO—O—R17-D or —CO—NH—R17-D group in which R17 is an alkylene group and D is a quaternary ammonium group;

A1 and B1 represent polymethylene groups containing from 2 to 20 carbon atoms, which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from a mineral or organic acid;

A1, R13 and R15 may form, with the two nitrogen atoms to which they are attached, a piperazine ring; moreover, if A1 denotes a saturated or unsaturated, linear or branched alkylene or hydroxyalkylene group, B1 may also denote a group (CH2)n-CO—D-OC—(CH2)p-

n and p are integers ranging from 2 to 20 approximately

in which D denotes:

a) a glycol residue of formula: —O—Z—O—, where Z denotes a linear or branched hydrocarbon-based radical or a group corresponding to one of the following formulae:

—(CH2-CH2-O)x-CH2-CH2-

—[CH2-CH(CH3)-O]y-CH2-CH(CH3)-

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based radical, or alternatively the divalent radical

—CH2-CH2-S—S—CH2-CH2-;

d) a ureylene group of formula: —NH—CO—NH—;

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers which are composed of repeating units conforming to the formula:

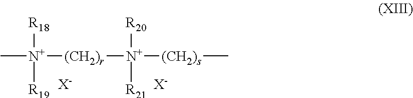

in which R18, R19, R20 and R21, which may be identical or different, denote an alkyl or hydroxyalkyl group having from about 1 to 4 carbon atoms, r and s are integers varying from 2 to 20 approximately, and X⁻ is an anion derived from a mineral or organic acid.

One particularly preferred compound of formula (XIII) is that for which R18, R19, R20 and R21 represent a methyl radical and r=3, s=6 and X=Cl, which is called Hexadimethrine chloride according to the INCl (CTFA) nomenclature.

(8) polyquaternary ammonium polymers composed of units of formula (XIV):

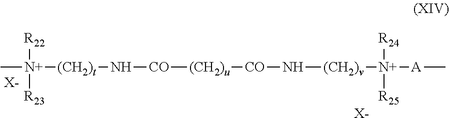

in which formula:

R22, R23, R24 and R25, which may be identical or different, represent a hydrogen atom or a methyl, ethyl, propyl, β-hydroxyethyl, β-hydroxypropyl or —CH2CH2(OCH2CH2)pOH radical,

in which p is equal to 0 or to an integer between 1 and 6, with the proviso that R22, R23, R24 and R25 do not simultaneously represent a hydrogen atom,

t and u, which may be identical or different, are integers between 1 and 6,

v is equal to 0 or to an integer between 1 and 34,

X⁻ denotes an anion such as a halide,

A denotes a radical of a dihalide or represents preferably —CH2-CH2-O—CH2-CH2-.

Such compounds are especially described in patent application EP-A-122 324.

Among these products, examples that may be mentioned include Mirapol® A 15, Mirapol® AD1, Mirapol® AZ1 and Mirapol®175 sold by the company Miranol.

(9) quaternary polymers of vinylpyrrolidone and of vinylimidazole, for instance the products sold under the names Luviquat® FC 905, FC 550 and FC 370 by the company BASF.

(10) cationic polysaccharides, especially cationic celluloses and galactomannan gums.

Among the cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French patent 1 492 597. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble monomer of quaternary ammonium, and described especially in U.S. Pat. No. 4,131,576, such as hydroxyalkylcelluloses, for instance hydroxymethyl-, hydroxyethyl- or hydroxypropyl-celluloses grafted, in particular, with a methacryloylethyltrimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Guar gums modified with a salt (e.g. chloride) of 2,3-epoxypropyltrimethylammonium are used, for example.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular mass may vary, for example, from 1500 to 10 000 and in particular from 2000 to 5000 approximately. Among these compounds, mention may be made especially of:
collagen hydrolysates bearing triethylammonium groups, such as the products sold under the name Quat-Pro E by the company Maybrook and referred to in the CTFA dictionary as Triethonium Hydrolyzed Collagen Ethosulfate;
collagen hydrolysates bearing trimethylammonium and trimethylstearylammonium chloride groups, sold under the name Quat-Pro S by the company Maybrook and referred to in the CTFA dictionary as Steartrimonium Hydrolyzed Collagen;
animal protein hydrolysates bearing trimethylbenzylammonium groups, such as the products sold under the name Crotein BTA by the company Croda and referred to in the CTFA dictionary as Benzyltrimonium hydrolyzed animal protein;
protein hydrolysates bearing on the polypeptide chain quaternary ammonium groups comprising at least one alkyl radical containing from 1 to 18 carbon atoms.

Among these protein hydrolysates, mention may be made, inter alia, of:
Croquat L in which the quaternary ammonium groups comprise a C12 alkyl group;
Croquat M in which the quaternary ammonium groups comprise C10-C18 alkyl groups;
Croquat S in which the quaternary ammonium groups comprise a C18 alkyl group;
Crotein Q in which the quaternary ammonium groups comprise at least one alkyl group containing from 1 to 18 carbon atoms.

These various products are sold by the company Croda.

Other quaternized proteins or hydrolysates are, for example, those corresponding to formula (XV):

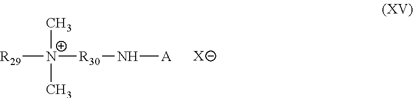

in which X⁻ is an anion of an organic or mineral acid, A denotes a protein residue derived from collagen protein hydrolysates, R29 denotes a lipophilic group comprising up to 30 carbon atoms, and R30 represents an alkylene group containing from 1 to 6 carbon atoms. Mention may be made, for example, of the product sold by the company lnolex under the name Lexein QX 3000, referred to in the CTFA dictionary as Cocotrimonium Collagen Hydrolysate.

Mention may also be made of quaternized plant proteins such as wheat, corn or soybean protein: as quaternized wheat protein, mention may be made of the products sold by the company Croda under the names Hydrotriticum WQ or QM, referred to in the CTFA dictionary as Cocodimonium hydrolysed wheat protein, Hydrotriticum QL referred to in the CTFA dictionary as Laurdimonium hydrolysed wheat protein, or Hydrotriticum QS, referred to in the CTFA dictionary as Steardimonium hydrolysed wheat protein.

Among all the cationic polymers that may be used in the context of the present invention, it is preferred to use cationic cyclopolymers, in particular dimethyldiallylammonium chloride homopolymers or copolymers, sold under the names Merquat 100, Merquat 550 and Merquat S by the company Nalco, quaternary polymers of vinylpyrrolidone and of vinylimidazole, and cationic polysaccharides, and mixtures thereof.

Among the organosiloxanes that may be used as conditioning agents in accordance with the present invention, mention may be made, in a non-limiting manner, of:

I. Volatile silicones:

These silicones have a boiling point of between 60° C. and 260° C. Among the silicones of this type that are mentioned are:

(i) cyclic silicones comprising from 3 to 7 and preferably 4 to 5 silicon atoms.

These are, for example, octamethylcyclotetrasiloxane sold under the name Volatile Silicone 7207® by the company Union Carbide or Silbione 70045 V2® by the company Rhône-Poulenc, decamethylcyclopentasiloxane sold under the name Volatile Silicone 7158® by the company Union Carbide, and Silbione 70045 V5® by the company Rhône-Poulenc, and mixtures thereof. Mention is also made of cyclopolymers of the dimethylsiloxane/methylalkylsiloxane type, such as Volatile Silicone FZ 3109® sold by the company Union Carbide, which is a dimethylsiloxane/methyloctylsiloxane cyclopolymer;

(ii) linear volatile silicones containing 2 to 9 silicon atoms and having a viscosity of less than or equal to 5×10⁻⁶ m²/s at 25° C.

Such a silicone is, for example, hexamethyldisiloxane sold under the name Silbione 70041 V0.65® by the company Rhône-Poulenc. This type of product is described in the article by Todd & Byers “Volatile silicone fluids for cosmetics”, Cosmetics and Toiletries, Vol. 91, Jan 76, pages 27 32.

II. Non-volatile silicones

These silicones are mainly constituted by polyalkylsiloxanes, polyarylsiloxanes, polyalkylarylsiloxanes and organomodified polysiloxanes, and mixtures thereof. They may be in the form of oils, gums and resins.

Among the polyalkylsiloxanes, mention may be made mainly of linear polydimethylsiloxanes with a viscosity of greater than 5×10⁻⁶ m²/s, and preferably less than 2.6 m²/s, i.e.:
containing trimethylsilyl end groups, for instance, in a non-limiting manner, the Silbione® oils of the 70047 series sold by the company Rhône-Poulenc, the oil Wacker Belsil DM 60000 from Wacker or certain Viscasil® products from the company General Electric,
containing trihydroxysilyl end groups of the 48 V® series from the company Rhône-Poulenc.

In this class of polyalkylsiloxanes, mention may also be made of the polyalkylsiloxanes sold by the company Goldschmidt under the names Abil Wax 9800® and Abil Wax 9801®, which are poly(C1-20)alkylsiloxanes.

Among these polyalkylarylsiloxanes, mention may be made of linear and/or branched polydimethylphenylsiloxanes and polydimethyldiphenylsiloxanes, with a viscosity from 10⁻⁵ to 5×10⁻² m²/s, for instance:
the oil Rhodorsil® 763 from Rhône-Poulenc,
the Silbione® oils of the 70641 series from Rhône-Poulenc, such as the oils Silbione 70641 V30® and Silbione 70641 V200®,
the product DC 556® Cosmetic Grade Fluid from Dow Corning,
the silicones of the PK series from Bayer, such as PK20®,
the silicones of the PN and PH series from Bayer, such as the products PN1000® and PH1000®;
certain oils of the SF series from General Electric, such as SF 1250®, SF 1265®, SF 1154® and SF 1023®.

The silicone gums in accordance with the present invention are polydiorganosiloxanes with a high number-average molecular mass of between 200 000 and 1 000 000, used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of compounds having the following structures:
poly[(dimethylsiloxane)/(methylvinylsiloxane)] gums,
poly[(dimethylsiloxane)/(diphenylsiloxane)] gums,
poly[(dihydrogenodimethylsiloxane)/(divinylsiloxane)] gums,
poly[(dimethylsiloxane)/(phenylmethylsiloxane)] gums,
poly[(dimethylsiloxane)/(diphenylsiloxane)/(methylvinylsiloxane)] gums.

Mention may be made of the gum Mirasil DM 300 000 from the company Rhodia. Examples that may also be mentioned, in a non-limiting manner, include the following mixtures:

1) mixtures formed from a polydimethylsiloxane hydroxylated at the end of the chain (dimethiconol according to the CTFA nomenclature), and of a cyclic polydimethylsiloxane (cyclomethicone according to the CTFA nomenclature), such as the products Q2 1401® or Dow Corning 1501 Fluid sold by the company Dow Corning;

2) mixtures formed from a polydimethylsiloxane gum with a cyclic silicone, such as the product SF 1214 Silicone Fluid® from General Electric, which is a gum SE 30® of MW 500 000 (—Mn) dissolved in SF 1202 Silicone Fluid® (decamethylcyclopentasiloxane);

3) mixtures of two PDMSs of different viscosities, especially of a PDMS gum and a PDMS oil, such as the products SF 1236® and CF 1241® from the company General Electric. The product SF 1236® is a mixture of an SE 30® gum defined above, with a viscosity of 20 m²/s, and of an SF 96® oil with a viscosity of 5×10⁶ m²/s (15% by weight of SE 30® gum and 85% by weight of SF 96® oil, relative to the total weight of the mixture).

The product CF 1241® is a mixture of an SE 30® gum (33% by weight) and of a PDMS (67% by weight), with a viscosity of 10⁻³ m2/s.

The organopolysiloxane resins that may be used in accordance with the invention are crosslinked siloxane systems containing the following units: R2SiO2/2, RSiO3/2 and SiO4/2 in which R represents a hydrocarbon group containing 1 to 6 carbon atoms or a phenyl group. Among these products, those particularly preferred are the ones in which R denotes a lower alkyl radical or a phenyl radical.

Among these resins, mention may be made of the product sold under the name Dow Corning 593® or those sold under the names Silicone Fluid SS 4230 and Silicone Fluid SS 4267 by the company General Electric, which are dimethyl/trimethylpolysiloxanes.

The organomodified silicones in accordance with the present invention are silicones as defined above, comprising in their general structure one or more organofunctional groups directly attached to the siloxane chain or attached via a hydrocarbon-based radical.

Mention is made, for example, of silicones comprising:

a) perfluoro groups such as trifluoroalkyls, for instance those sold by the company General Electric under the names FF.150 Fluorosilicone Fluid® or by the company Shin-Etsu under the names X-22-819®, X-22-82®, X-22-821® and X-22-822®;

b) hydroxyacylamino groups, for instance those described in patent application EP 0 342 834 and in particular the silicone sold by the company Dow Corning under the name Q2-8413®;

c) thiol groups, as in the silicones X 2-8360® from the company Dow Corning or GP 72A® and GP 71® from Genesee;

d) non-quaternized amine groups, such as GP 4 Silicone Fluid® from Genesee, GP 7100® from Genesee, Q2 8220® from Dow Corning, AFL 40® from Union Carbide or the silicone known as Amodimethicone in the CTFA dictionary;

e) carboxylate groups, for instance the products described in patent EP 186 507 from Chisso Corporation;

f) hydroxylated groups, for instance polyorganosiloxanes containing a hydroxyalkyl function, for example those described in patent application FR 85/16334, corresponding to formula (XVI) below:

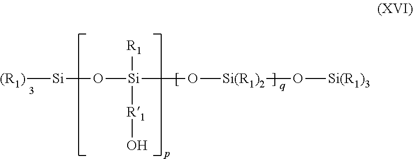

in which:
the radicals R1, which may be identical or different, are chosen from methyl and phenyl radicals, at least 60 mol % of the radicals R1 being methyl radicals;
the radical R¹1 is a C2-C18 hydrocarbon-based divalent alkylene chain unit;
p is between 1 and 30 inclusive;
q is between 1 and 150 inclusive.

Mention may be made most particularly of the product sold by Dow Corning under the name DC 190;

g) alkoxylated groups, for instance in the silicone copolymer F 755® from SWS Silicones and the products Abil Wax 2428®, Abil Wax 2434® and Abil Wax 2440® from the company Goldschmidt;

h) acyloxyalkyl groups, for instance the polyorganopolysiloxanes described in patent application FR 88/17433, corresponding to formula (XVII) below:

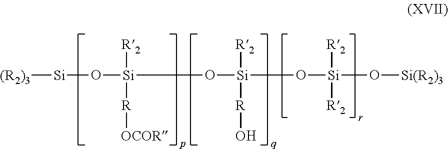

in which:
R2 denotes methyl, phenyl, OCOR″ or hydroxyl, only one of the radicals R2 per silicon atom may be OH;
R′2 denotes methyl or phenyl, at least 60 mol% of all the radicals R2 and R′2 are methyl;
R″ denotes C8-C20 alkyl or alkenyl;
R denotes a linear or branched C2-C18 divalent hydrocarbon-based alkylene;
r is between 1 and 120 inclusive;
p is between 1 and 30 inclusive;
q is 0 or is less than 0.5 p, p+q being between 1 and 30 inclusive:

the polyorganosiloxanes of formula (XVII) may contain groups:

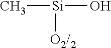

in proportions not exceeding 15% of the sum p+q+r;

i) quaternary ammonium groups, as in the products X2 8108® and X2 8109® and the product Abil K 3270® from the company Goldschmidt;

j) amphoteric or betaine groups, as in the product sold by the company Goldschmidt under the name Abil B 9950®;

k) bisulfite groups, as in the products sold by the company Goldschmidt under the names Abil S 201® and Abil S 255®.

l) polyethyleneoxy and/or polypropyleneoxy groups optionally comprising C6-C24 alkyl groups, such as the products known as dimethicone copolyol sold by the company Dow Corning under the name DC 1248 or the oils Silwet L 722, L 7500, L 77 and L 711 by the company Union Carbide, and the (C12)alkylmethicone copolyol sold by the company Dow Corning under the name Q2 5200;

According to the invention, silicones comprising a polysiloxane portion and a portion formed from a non-silicone organic chain, one of the two portions forming the main chain of the polymer and the other being grafted onto the said main chain, may also be used. These polymers are described, for example, in patent applications EP-A-412 704, EP-A-412 707, EP-A-640 105 and WO 95/00578, EP-A-582 152 and WO 93/23009 and patents U.S. Pat. No. 4,693,935, U.S. Pat. No. 4,728,571 and U.S. Pat. No. 4,972,037. These polymers are preferably anionic or nonionic.

Such polymers are, for example, copolymers that may be obtained by radical polymerization from the monomer mixture formed from:

a) 50 to 90% by weight of tert-butyl acrylate;

b) 0 to 40% by weight of acrylic acid;

c) 5 to 40% by weight of a silicone macromer of formula:

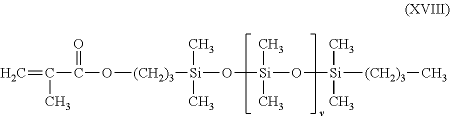

with v being a number ranging from 5 to 700; the weight percentages being calculated relative to the total weight of the monomers.

Other examples of grafted silicone polymers are especially polydimethylsiloxanes (PDMSs) onto which are grafted, via a connecting unit of thiopropylene type, mixed polymer units of the poly(meth)acrylic acid type and of the polyalkyl (meth)acrylate type and polydimethylsiloxanes (PDMSs) onto which are grafted, via a connecting unit of thiopropylene type, polymer units of the polyisobutyl (meth)acrylate type.

According to the invention, all the silicones may also be used in the form of emulsions, nanoemulsions or microemulsions.

The polyorganosiloxanes that are particularly preferred in accordance with the invention are:
non-volatile silicones chosen from the family of polydialkylsiloxanes containing trimethylsilyl end groups, such as oils with a viscosity of between 0.2 and 2.5 m²/s at 25° C., such as the oils of the series DC200 from Dow Corning, in particular the product with a viscosity of 60 000 cSt, of the series Silbione 70047 sold by the company Rhodia Chimie, silicone gums and polydialkylsiloxanes containing dimethylsilanol end groups such as dimethiconols, or polyalkylarylsiloxanes such as the oil Silbione 70641 V 200 sold by the company Rhodia Chimie;
polysiloxanes containing amine groups such as amodimethicones or trimethylsilyl amodimethicones;

The viscosities of the silicones may especially be determined by the standard ASTM D445-97 (viscometry).

When the conditioning agent of the composition according to the invention is a hydrocarbon, it is a linear or branched C8-C300 hydrocarbon.

Among the hydrocarbons that are liquid at room temperature corresponding to this definition mention may be made especially of isododecane, isohexadecane and isomers thereof (such as 2,2,4,4,6,6-heptamethylnonane), isoeicosane, isotetracosane, isomers of the said compounds, n-nonadecane, n-dodecane, n-undecane, n-tridecane and n-pentadecane, and mixtures of these hydrocarbons. Use is preferably made according to the invention of isododecane or an isomer thereof.

When the conditioning agent is a fatty alcohol, it is of the linear or branched, saturated or unsaturated C8-C30 type. Among the latter agents, examples that may be mentioned include 2-butyloctanol, lauryl alcohol, oleyl alcohol, 2-octyldodecanol, isocetyl alcohol, isostearyl alcohol and behenyl alcohol, and mixtures thereof.

When the conditioning agent is a fatty ester, it may be either an ester of a C8-C30 fatty acid and of a C1-C30 alcohol, or an ester of a C1-C7 acid or diacid and of a C8-C30 fatty alcohol, or an ester of a C8-C30 fatty acid and of a C8-C30 fatty alcohol.

Among these esters, examples that may be mentioned include ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate, dioctyl malate, myristyl myristate and cetyl palmitate, and mixtures thereof.

The ceramides or ceramide analogues, such as glycoceramides, which may be used as conditioning agents in the compositions according to the invention are known per se and are natural or synthetic molecules that may correspond to the general formula (XIX) below:

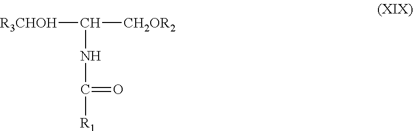

in which:
R1 denotes a linear or branched, saturated or unsaturated alkyl radical, derived from C14-C30 fatty acids, this radical possibly being substituted with a hydroxyl group or a hydroxyl group in the omega position esterified with a saturated or unsaturated C16-C30 fatty acid;
R2 denotes a hydrogen atom or a radical (glycosyl)n, (galactosyl)m or sulfogalactosyl, in which n is an integer ranging from 1 to 4 and m is an integer ranging from 1 to 8;
R3 denotes a saturated or unsaturated C15-C26 hydrocarbon-based radical in the alpha position, this radical possibly being substituted with one or more C1-C14 alkyl radicals;

it being understood that, in the case of these natural ceramides or glycoceramides, R3 may also denote a C15-C26 alpha-hydroxyalkyl radical, the hydroxyl group being optionally esterified with a C16-C30 alpha-hydroxy acid.

The ceramides that are preferred in the context of the present invention are those described by Downing in Arch. Dermatol, Vol. 123, 1381-1384, 1987, or those described in French patent FR 2 673 179.

The ceramide(s) that are more particularly preferred according to the invention are the compounds for which R1 denotes a saturated or unsaturated alkyl derived from C16-C22 fatty acids; R2 denotes a hydrogen atom and R3 denotes a saturated linear C15 radical.

Such compounds are, for example:
N-linoleoyldihydrosphingosine,
N-oleoyldihydrosphingosine,
N-palmitoyldihydrosphingosine,
N-stearoyldihydrosphingosine,
N-behenoyldihydrosphingosine,

or mixtures of these compounds.

Even more preferentially, used is made of ceramides for which R1 denotes a saturated or unsaturated alkyl radical derived from fatty acids; R2 denotes a galactosyl or sulfogalactosyl radical; and R3 denotes a group —CH═CH—(CH2)12-CH3.

By way of example, mention may be made of the product formed from a mixture of these compounds, sold under the trade name Glycocer by the company Waitaki International Biosciences.

Among all these conditioning agents, use is preferably made of one or more conditioning agents chosen from organosiloxanes and cationic polymers.

The cosmetic composition according to the invention preferably contains from 0.01% to 20% by weight and more preferentially from 0.05% to 10% by weight of conditioning agent(s), other than the cationic surfactants (ii), relative to the total weight of the composition.

The composition according to the invention may also comprise one or more standard additives that are well known in the art, other than the compounds defined previously. As examples of additives that may be used according to the invention, mention may be made of silanes, anionic surfactants, amphoteric or zwitterionic surfactants, nonionic surfactants, proteins, protein hydrolysates, vitamins, reducing agents, plasticizers, softeners, antifoams, moisturizers, pigments, clays, mineral fillers, UV-screening agents, mineral colloids, peptizers, solubilizers, fragrances, preserving agents, nacreous agents, propellants and mineral or organic thickeners; these additives being different from the compounds defined hereinabove.

A person skilled in the art will take care to select the optional additive(s) and the amount thereof such that they do not harm the properties of the compositions of the present invention.

The additive(s) are generally present in the composition according to the invention in an amount ranging from 0 to 20% by weight relative to the total weight of the composition.

The compositions according to the invention may be in any galenical form conventionally used for topical application and especially in the form of aqueous or aqueous-alcoholic solutions, oil-in-water (O/W) or water-in-oil (W/O) or multiple emulsions (triple: W/O/W or O/WIO), aqueous gels, or dispersions of a fatty phase in an aqueous phase by means of spherules, these spherules possibly being polymer nanoparticles such as nanospheres and nanocapsules or lipid vesicles of ionic and/or nonionic type (liposomes, niosomes or oleosomes). These compositions are prepared according to the usual methods.

In addition, the compositions used according to the invention may be more or less fluid and may have the appearance of a white or coloured cream, a pomade, a milk, a lotion, a serum, a paste or a mousse. They may be optionally applied to the keratin materials in aerosol form. They may also be in solid form, for example in the form of a stick.

Needless to say, a person skilled in the art will take care to select the optional compound(s) to be added to the composition according to the invention, such that the advantageous properties intrinsically associated with the composition in accordance with the invention are not, or are not substantially, adversely affected by the envisaged addition(s).

Another subject of the invention consists of a cosmetic process for treating the scalp and keratin fibres, in particular human keratin fibres such as the hair, comprising the application of a composition according to the invention to the scalp and the said keratin fibres. After an optional leave-in time preferably ranging from 1 to 15 minutes, the composition is then preferably rinsed out with water. Preferably, the said process is intended for conditioning the said keratin fibres and/or for an antidandruff treatment for eliminating and/or reducing dandruff, and especially that caused by yeasts of the genus Malassezia.

A subject of the invention is also the use of a composition according to the invention for caring for keratin fibres, in particular human keratin fibres such as the hair.

A subject thereof is also the use of a composition according to the invention for antidandruff treatment for eliminating and/or reducing dandruff, especially that caused by yeasts of the genus Malassezia.

The examples that follow are intended to illustrate the invention, without, however, having any limiting nature.

### EXAMPLE 1

Two antidandruff haircare products having the following composition were prepared:

| Ellagic acid | 1 | | 0.7 | |
|---|---|---|---|---|
| Dipalmitoylethylhydroxyethyl- | 0.3 | g AM | 1.8 | g AM |
| methylammonium methosulfate at | | | | |
| 30% by weight in cetylstearyl | | | | |
| alcohol (Dehyquart F30 - Cognis) | | | | |
| Behenyltrimethylammonium chloride | 0.48 | g AM | | |
| at 80% by weight of active | | | | |
| material (Genamin KDMP - Clariant) | | | | |
| Cetyltrimethylammonium chloride | 0.625 | g AM | | |
| at 25% by weight of active material | | | | |
| (Dehyquart A OR from Cognis) | | | | |
| Polydimethylsiloxane containing | 1.54 | g AM | | |
| aminoethyl iminopropyl groups, | | | | |
| as a cationic emulsion at 60% by | | | | |
| weight in water (Dow Corning 2- | | | | |
| 8299 Cationic Emulsion from | | | | |
| Dow Corning) | | | | |
| Liquid jojoba wax | 0.1 | g | | |
| Cetyl alcohol | 3.7 | g | 4.5 | g |
| Myristyl alcohol, sold under the | 0.4 | g | | |
| trade name Nacol 14-98 by the | | | | |
| company Sasol | | | | |
| Hydroxyethylcellulose (Natrosol | 0.25 | g | 0.25 | g |
| 250 HHR sold by Aqualon) | | | | |
| Myristyl/cetyl/stearyl myristate/ | 0.8 | g | 0.8 | g |
| palmitate/stearate mixture | | | | |
| (Crodamol MS-PA-(mh) from Croda) | | | | |
| Citric acid | 0.03 | g | 0.03 | g |
| Fragrance, preserving agents | qs | | qs | |
| Deionized water qs | qs 100 | g | qs 100 | g |

The compositions are prepared according to the following procedure:

Water, citric acid, ellagic acid, preserving agents, hydroxyethylcellulose and behenyltrimethylammonium chloride, if it is present, are placed in a container. The mixture is heated to 50° C. The cetyltrimethylammonium chloride, if it is present, is added. The mixture is heated to 60° C. The cetyl alcohol, the myristyl alcohol, if it is present, the mixture of esters, the liquid jojoba wax, if it is present, and the dipalmitoylethylhydroxyethylmethylammonium methosulfate are added. The mixture is heated to 70° C. and maintained at this temperature until the solid fatty substances have melted. It is cooled to 30° C.

A fine, homogeneous dispersion of ellagic acid in the composition is obtained. The composition is stable over time (no macroscopic change after two months of storage at room temperature (25° C.) or at 45° C.).

When applied to the hair and the scalp, this care product promotes the spreading of the ellagic acid onto the scalp, which promotes the antidandruff efficacy. The hair shows during and after the treatment good cosmetic properties especially in terms of softness, smoothness and disentangling.

### EXAMPLE 2

An antidandruff haircare product having the following composition was prepared:

| Ellagic acid | 1 | g |
|---|---|---|
| Behenyltrimethylammonium chloride at 80% by | 1.6 | g AM |
| weight of active material (Genamin KDMP - | | |
| Clariant) | | |
| Methylalkylalkylamidoethylimidazolium methosulfate | 2.5 | g AM |
| as a 75% solution in propylene glycol (Varisoft W | | |
| 575 PG - Evonik Goldschmidt) | | |
| Candelilla wax | 0.3 | g |
| Stearyl alcohol | 2 | g |
| Isopropyt myristate | 0.8 | g |
| C12/C15 alkyl benzoate (Tegosoft TN - Evonik | 5 | g |
| Goldschmidt) | | |
| Sorbitan monolaurate with 20 mol of ethylene | 0.3 | |
| oxide (Tween 20 LQ - Croda) | | |
| Potato starch modified with 2- | 2.6 | g AM |
| chloroethylaminodipropionic acid at 86% in water | | |
| and neutralized with sodium hydroxide (Structure | | |
| Solanace - Akzo Nobel) | | |
| Fragrance, preserving agents | qs | |
| Deionized water qs | qs 100 | g |

A fine, homogeneous dispersion of ellagic acid in the composition is obtained. The composition is stable over time (no macroscopic change after two months of storage at room temperature (25° C.) or at 45° C.).

When applied to the hair and the scalp, this care product promotes the spreading of the ellagic acid onto the scalp, which promotes the antidandruff efficacy. The hair shows during and after the treatment good cosmetic properties especially in terms of softness, smoothness and disentangling.

1-10. (canceled)

11. A cosmetic composition comprising, in a cosmetically acceptable medium:
(i) from 0.1% to 10% by weight, relative to the total weight of the composition, of at least one compound chosen from ellagic acid, ethers of ellagic acid, ellagic acid salts, and ethers of ellagic acid salts; and
(ii) at least one cationic surfactant comprising one or more permanent positive charges chosen from
the quaternary ammonium salts corresponding to the general formula (II) below: 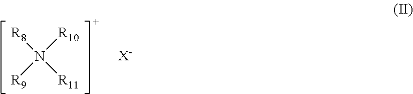
in which the radicals R₈ to R₁₁, which may be identical or different, represent a linear or branched aliphatic radical, comprising from 1 to 30 carbon atoms and possibly comprising heteroatoms, or alternatively an aromatic radical, at least one of the radicals R₈ to R₁₁ comprising from 8 to 30 carbon atoms; and
quaternary ammonium salts containing at least one ester function.

12. The composition according to claim 11, wherein the at least one compound (i) is chosen from alkali metal or alkaline-earth metal salts of ellagic acid and ethers thereof.

13. The composition according to claim 11, wherein the at least one compound (i) is chosen from sodium, potassium, calcium, and magnesium salts of ellagic acid and ethers thereof.

14. The composition according to claim 11, wherein the at least one compound (i) is an ellagic acid ether or ellagic acid salt ether chosen from mono-, di-, tri- or polyethers obtained by etherification of one or more hydroxyl groups of ellagic acid with one or more groups chosen from C₂-C₂₀ alkyl groups, polyoxyalkylene groups and groups derived from one or more mono- or polysaccharides.

15. The composition according to claim 11, wherein the at least one compound (i) is ellagic acid or a salt thereof.

16. The composition according to claim 11, wherein the at least one compound (i) is present in a an amount ranging from about 0.1% to about 5% by weight, relative to the total weight of the composition.

17. The composition according to claim 11, wherein the at least one compound (i) is present in a an amount ranging from about 0.2% to about 2% by weight, relative to the total weight of the composition.

18. The composition according to any claim 11, wherein the at least one cationic surfactant is chosen from cetyltrimethylammonium salts, behenyltrimethylammonium salts, dipalmitoylethylhydroxyethylmethylammonium salts, distearoylethylhydroxyethylmethylammonium salts and methyl(C₉-C₁₉)alkyl(C₁₀-C₂₀)alkylamidoethylimidazolium salts, and mixtures thereof.

19. The composition according to claim 11, wherein the at least one cationic surfactant is present in an amount ranging from about 0.01% to about 15% by weight relative to the total weight of the composition.

20. The composition according to claim 11, wherein the at least one cationic surfactant is present in an amount ranging from about 0.1% to about 10% by weight, relative to the total weight of the composition.

21. The composition according to claim 11, wherein the at least one cationic surfactant is present in an amount ranging from about 0.2% to about 5% by weight, relative to the total weight of the composition.

22. The composition according to clam 11, wherein the weight ratio of the at least one cationic surfactant (ii)/at least one compound (i) ranges from about 0.1 to about 50.

23. The composition according to clam 11, wherein the weight ratio of the at least one cationic surfactant (ii)/at least one compound (i) ranges from about 0.5 to about 25.

24. The composition according to clam 11, wherein the weight ratio of the at least one cationic surfactant (ii)/at least one compound (i) ranges from about 1 to about 10.

25. A method of eliminating and/or reducing dandruff, said method comprising applying, to keratinous fibers, an effective amount of a composition comprising, in a cosmetically acceptable medium:
(i) from 0.1% to 10% by weight, relative to the total weight of the composition, of at least one compound chosen from ellagic acid, ethers thereof, ellagic acid salts, and ethers thereof, and
(ii) at least one cationic surfactant comprising one or more permanent positive charges chosen from
the quaternary ammonium salts corresponding to the general formula (II) below: 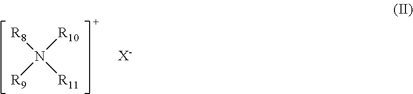
in which the radicals R₈ to R₁₁, which may be identical or different, represent a linear or branched aliphatic radical, comprising from 1 to 30 carbon atoms and possibly comprising heteroatoms, or alternatively an aromatic radical, at least one of the radicals R₈ to R₁₁ comprising from 8 to 30 carbon atoms; and
quaternary ammonium salts containing at least one ester function.

26. A non-therapeutic method of treating keratinous fibers, said method comprising applying, to said keratinous fibers, a composition comprising, in a cosmetically acceptable medium:
(i) from 0.1% to 10% by weight, relative to the total weight of the composition, of at least one compound chosen from ellagic acid, ethers thereof, ellagic acid salts, and ethers thereof, and
(ii) at least one cationic surfactant comprising one or more permanent positive charges chosen from
the quaternary ammonium salts corresponding to the general formula (II) below: 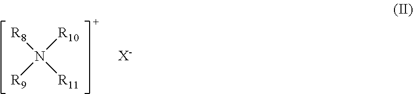
in which the radicals R₈ to R₁₁, which may be identical or different, represent a linear or branched aliphatic radical, comprising from 1 to 30 carbon atoms and possibly comprising heteroatoms, or alternatively an aromatic radical, at least one of the radicals R₈ to R₁₁ comprising from 8 to 30 carbon atoms; and
quaternary ammonium salts containing at least one ester function.
